(19) United States
(12) Patent Application Publication
(10) Pub. No.: US 2010/0041776 A1
(43) Pub. Date: Feb. 18, 2010

(54) GLYCEROL CONVERSION INTO CLEAN AND RENEWABLE LIQUID FUEL

(75) Inventors: Albin Czernichowski, Orleans (FR); Jan Czernichowski, Orleans (FR); Caroline Czernichowski, Layton, UT (US)

Correspondence Address: Zies Widerman & Malek, P.L., 202 N. Harbor City Blvd, Suite #101, Melbourne, FL, 32935, US

(73) Assignee: Florida Syngas, Grant, FL (US)

(21) Appl. No.: 12/497,156

(22) Filed: Jul. 2, 2009

(30) Foreign Application Priority Data
Jul. 3, 2008 (FR) 08 03755

(51) Int. Cl. C07C 27/00

(52) U.S. Cl. 518/700

(57) ABSTRACT

A technique of cooling of a reactor with a fixed catalytic bed for exothermic catalytic syntheses, such as the Fischer-Tropsch (FT) synthesis of liquid fuels by selective hydrogenation of the monoxide of carbon (CO) by the hydrogen (H₂), includes mixing of these two gases being called “synthesis gas” or “syngas”, and is characterized by the fact that the heat of such synthesis is taken away by a heat medium of non-fossil origin and having a high boiling point in comparison with water and that at least a part of the resultant vapors of such boiling heat medium is extracted from the reactor to feed, as a carbonaceous matter, another reactor, called “reformer”, in which this vapor is converted into syngas to feed the aforesaid reactor of exothermic synthesis.

### SUMMARY

The method is composed of mainly two integrated Steps:
C. Conversion of the glycerol vapor into the synthesis gas for the Fischer-Tropsch (FT) synthesis of liquid hydrocarbons, and
D. Vaporization of the liquid glycerol used as cooling medium for the same exothermic FT process.

Use of the glycerol as cooling fluid (Step B) allows an increase of the operational temperature of the multiple-tube classical FT reactors without an increase of pressure inside the vessel where these tubes are lodged and cooled. Consequently, some specific high-temperature, high-output and reduced-poisoning FT catalysts can be used. The increase of temperature of cooling medium also allows augmenting the energy efficiency of thermodynamic machines converting FT waste heat flux into mechanical or electrical power. Another advantage of the glycerol used as heat medium is linked to its abundance due to the development of the biodiesel industry, which produces growing quantities of waste glycerol. Further to a surplus heat produced during the conversion of the glycerol vapor into syngas in the Step A, it becomes possible to include an additional integrated Step C:

Separation of salts and soaps in waste glycerol by an evaporation of all volatile molecules, which gives vapors that can be considered as plant/vegetal or animal carbon feed for the Step A and/or as a source of pure glycerol (after selective condensation of the vapors) to feed the Step B.

### BRIEF DESCRIPTION OF THE DRAWINGS

FIG. 1 is a schematic diagram illustrating an integrated solution of circulation of matter and of energy according to the present invention.

FIG. 2 is a schematic diagram illustrating a two-serial-zone apparatus to convert the glycerol into synthesis gas according to the present invention.

FIG. 3 is a schematic diagram of a plasma head according to the present invention.

FIG. 4 is a graphical illustration of pressure (in absolute bar) of saturating water steam and of the glycerol according to “Handbook of Chemistry and Physics, 81st and 44th Edition”.

### DESCRIPTION

The present invention concerns a new method and its implementation for the conversion of the glycerol into a liquid fuel. The method is composed of three integrated steps:
D. Conversion of the glycerol vapors into the synthesis gas devoted for the Fischer-Tropsch (FT) synthesis of hydrocarbons,
E. Vaporization of the liquid glycerol used as the exothermic FT cooling medium, and,
F. Separation of salts and soaps present in waste glycerol by an evaporation of all volatile molecules using residual heat produced in step A.

Tubular reactors with fixed bed are well-known equipment to lead chemical reactions accompanied with a strong thermal effect. It is case, for example, of FT synthesis reactors where grains of a specific catalyst speed up a reaction of the selective hydrogenation of carbon monoxide (CO) by hydrogen (H₂), the reaction that gives liquid hydrocarbons, according to schemas:

n CO+(2n+1)H₂ [symbol] CₙH₂ₙ₊₂+n H₂O (1)

n CO+2n H₂—CₙH₂ₙ+n H₂O (2)

2n CO+n H₂—CₙH₂ₙ+n CO₂ (3)

These reactions are strongly exothermic. For example, when 3 moles of the mixture H₂/CO (called “synthesis gas” or “syngas”), in molar proportions close to 2, are completely converted to the decane (C₁₀H₂₂), the standard enthalpy of reaction is equal to −161 kJ (298 K and 101 kPa). It corresponds to −80.5 kJ per mole of converted CO. Some 19.4 cm³ (14 g) of liquid hydrocarbon are then obtained. In reality, this synthesis in the presence of a catalyst based on cobalt (Co) gives a mixture of paraffins and of iso-paraffins characterized by a number of carbons n being generally between 5 and 25 by molecule. One notes a complete absence of sulfur, nitrogen, and aromatic hydrocarbons in the product that is indeed more and more searched as very clean fuel of substitution instead of current fuels coming from the crude oil.

Other catalysts are also offered for an industrial use. For example, catalysts based on iron are not expensive and easy to use.

The conditions of synthesis FT of hydrocarbons are generally well known to those skilled in the art. Temperatures can be between 150 and 380° C. Pressures are normally included between 0.5 and 6 MPa. In a general way, the increase of temperature augments productiveness. Some modern catalysts can just require working on high temperatures.

During our tests (scale of 5 liters with our catalyst based on iron), we were confronted with several problems that encouraged us to search means and methods to solve them. Processes (1-3) are rather slow and very delicate to lead. They ask for stability of pressure, flow rate and especially of temperature. That is why an isothermal regime for all volume of the catalyst must imperatively be assured. Otherwise, a grain of badly cooled catalyst would quickly heat-up because of exothermic reactions (1-3), what would cause a swing of these useful reactions towards a parasitic reaction, also exothermic

2CO═C+CO₂ (4)

with a deactivation of the grain by an accumulation of soot on the active surface of the catalyst.

Let us return to our calculations based only on the typical paraffin C₁₀ (decane). To produce 1 barrel (159 liters) of this paraffin it is necessary to convert theoretically 550 m³(n) of syngas, with a heat release of 1.3 GJ. A reactor producing 1 bbd (barrel a day) presents therefore a thermal power of 15 kW. This flux of energy is produced between 170 and 230° C., temperature of the FT synthesis using a catalyst based on the cobalt and put in a fixed bed, or even up to 380° C. for some catalysts based on the iron.

Tubes sheltering the catalyst FT, whatever is the reactor with fixed bed, are in vertical position with the syngas and the products of synthesis passing downward. It imposes a limitation of height on 5-12 m for these classical reactors of type “beam of thousand tubes inside a big vessel”. To put more and more tubes in the single vessel, it would be necessary to augment the diameter of the vessel and, therefore, the thickness of its walls (condition of its pressure resistance).

A classical multiple-tube reactor is cooled by water and/or steam under pressure, this heat medium being between the tubes, the whole being put together in this big metallic cylinder (vessel) under a high pressure of the medium and provided with entries and exits (reactants, products, heat medium). The tubes of the reactor are habitually fabricated from steel of an internal diameter from 20 to 80 mm with walls from 2.5 to 10 mm in thickness. A reactor of industrial size contains thousands of tubes, every tube having ends welded individually in a collector of syngas entrance on the one side and in an exit collector of products and of reactants to be regenerated across on the other side.

A current crisis of availability and high prices of liquid fuels (gasoline, diesel, and kerosene) reinforces the usefulness of the “Gas-to-Liquids” (GTL) technique of the generation of synthetic liquid fuels (“Synfuel” or “Syncrude”) based on process in two Operations:
III. Production of gas of synthesis (Syngas) from the natural gas,
IV. FT synthesis of liquid fuels from the syngas.

However, because of failing reserves and increasing prices of the natural gas, one perceives the emergence of GTL technologies based on syngas generation from vegetable/plant oils as well as waste liquids or vapors coming from the agro-industry or from other sectors. In the description that follows, we take the glycerol as a model of such liquids, to illustrate here-presented process and linked apparatuses.

The glycerol C₃H₅(OH)₃ (called also “glycerine” or “glycerin”) was considered in the past as a noble product, with a variety of applications at a relatively small scale. At present it becomes a co-product (almost a waste) got during the production of the biodiesel by the transesterification of vegetable oils and greases with the methanol in the presence of a catalyst. About 10% of oil (or of grease) is converted into glycerol during such process. Worldwide industry produces millions of gallons of glycerine per year and this quantity augments fast with an impressive growth of the production of biodiesel. It saturates the worldwide market of the glycerol and strongly diminishes its price as a result. Let us add that the glycerine burns hardly and cannot therefore be considered as fuel.

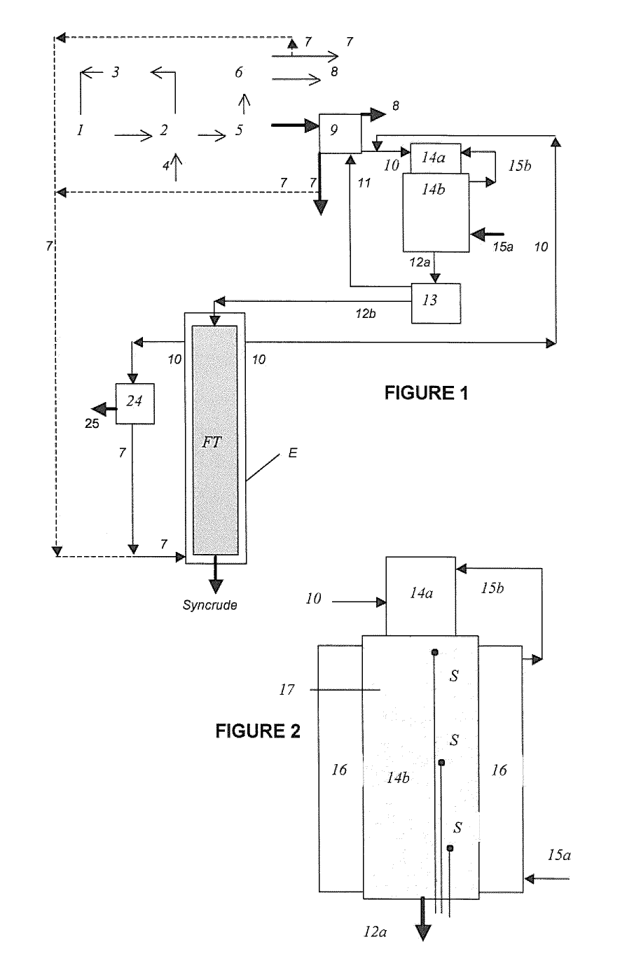

One of the most important uses of glycerol would be its gasification. The resultant syngas would then be used as a gaseous fuel, or better, as a reactant for the GTL process. In addition, the liquid glycerol can be used as heat medium in the FT process and the so-generated glycerol vapor can be considered to be reactant for the syngas generation. The present invention shows all points of such integrated conversion of glycerine. The FIG. 1 presents a general diagram of invention.

Box 1 represents a biodiesel plant. The co-produced glycerine 2 contains salts, organic liquids, and water. In an operation prior to all processes of its purification, filtering and/or centrifugation take the majority of solids away. From the remaining liquid only the unreacted methanol 3 is considered as a valuable compound for the biodiesel plant. It is therefore taken away of 2 by adding a low temperature heat 4 (the methanol has a boiling point at atmospheric pressure equal to only 65° C.). Very often this operation is carried out at the biodiesel plant.

The resultant raw glycerine 5 still contains different soluble salts, organic liquids, and some water. The final purification 6, if carried out by the operator of the biodiesel plant, is typically fulfilled by a vacuum distillation followed by a discoloration by activated charcoal or an ion exchange, followed by drying to take water away. Such distillation is very expensive, mainly because of the power consumption. The resultant pure glycerol 7 can be sold . . . if it finds a client. An ultimate mixture of salts and persistent soaps 8 is going to be treated for the recovery of energy and some salts.

Although such pure glycerol 7 is acceptable for our process installed in a separated site—it would be preferable to install the process near the biodiesel plant 1 for a better integration of heat and matter. Integration and valorization of the waste glycerine can also be made at a soap plant, where the same raw glycerin is found and where the problems that come are also the same.

Let us consider now the liquid 5 to be an abundant and cheap raw matter for the physical and chemical treatment according to the present invention. This time, we accept almost any glycerine, containing water, alcohols, hydrocarbons, organic acids, ketones, aldehydes, esters, and any variety of molecules composed of carbon (C), hydrogen (H), oxygen (O), and nitrogen (N)—except metals as Na, K, Ca, etc. In fact, we accept as reactants any carbonaceous liquid that does not contain minerals. More precisely, we take as entry matter a liquid, which does not contain persistent ashes by more of 0.1 g per liter after its treatment at minimum 1100° C. in the oxidizing atmosphere. To take away any surplus of ashes and minerals, we apply therefore a batch-type distillation 9 in which the raw liquid 5 is heated and completely evaporated by giving vapor 10, while minerals, salts and other ash precursors are taken away as the residue 8. The apparatus of distillation 9 can also include a dosing pump to control liquid flow rate.

We provide heat 11 for such atmospheric distillation (or at another pressure which can go up to 10 bar). This heat comes from the hot syngas 12a (at 350 to 500° C.) that cools in a heat exchanger 13. This hot syngas is produced by catalytic partial oxidation (POX) of vapor 10 in a two-zone apparatus 14a and 14b called “reformer”. Downstream from the exchanger 13, we get a relatively cold syngas 12b, similar to the syngas already mentioned in the Operation I of GTL process. The syngas presents a mixture of hydrogen (H₂), carbon oxide (CO), and carbon dioxide (CO₂). This mixture can contain some light hydrocarbons. These gases can be diluted in the nitrogen (N₂) if air is used as a source of oxygen called “oxidizer” 15a and 15b for this POX process.

The two-serial-zone apparatus to convert the glycerol into syngas is schematically presented on the FIG. 2. It includes a head, preferably with plasma, 14a (first zone) followed by a catalytic reformer 14b (second zone, called also “post-plasma zone”). A jacked 16 with an entrance and an exit can encircle the zone 14b. The oxidizer 15a at an ambient temperature (the first temperature) enters the jacket 16, warms up in it and exits as hot oxidizer 15b by an exit at a second temperature included between about 250 and 350° C. The zone 14b from the reformer contains a specific catalyst 17 that helps the POX reaction converting completely a carbonaceous vapor (like the glycerol vapor) into syngas. A multiplicity of temperature probes S can be included to control temperatures on the whole of the zone post plasma of the reformer.

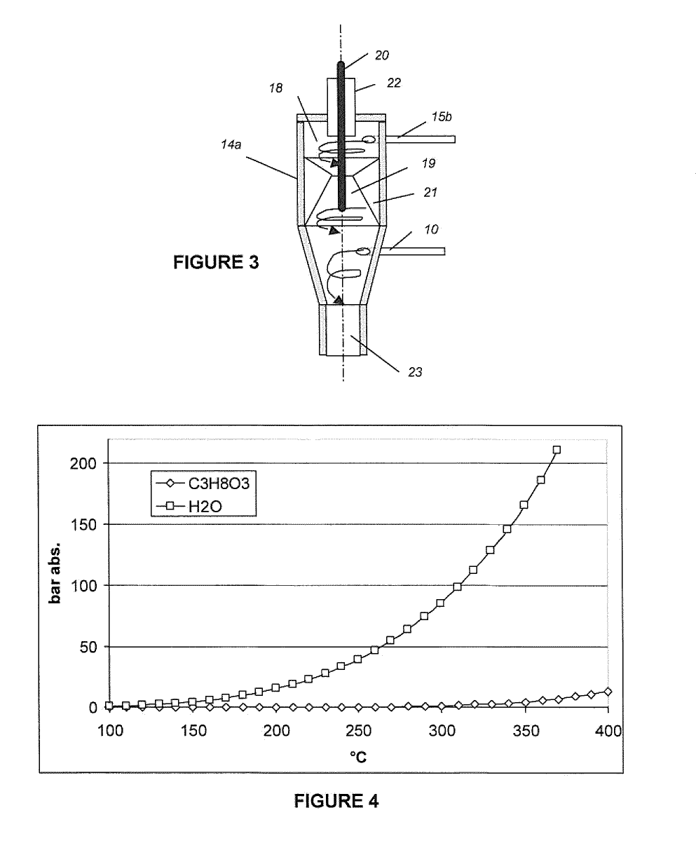

The plasma head 14a introduced in detail on the FIG. 3 includes preferably a zone of vortex 18 and a plasma zone 19, put downstream from the vortex zone. The plasma head also includes, preferably, an arrival of an oxidizer 15b adjacent to the upper portion of the vortex zone. The plasma head can also include a baffle adjacent to the oxidizer entry. The baffle can have an angle predetermined to cause the torodial movement of the oxidizer in the zone of vortex. The plasma head can also include a high voltage electrode 20 put in the vortex zone. An electrical discharge strikes between this electrode and another electrode 21 at the ground potential. The electrode 20 is electrically insulated from the head 14a by a ceramic material 22. The plasma head can also include a metallic insert playing the role of electrode 21, encircling a medial portion of the high voltage electrode 20. A form of hourglass can define internal portions of this metallic insert 21. Electrical discharge glides and turns between these electrodes at the potential difference included between 6 and 25 kV about. The plasma head also contains a vortex of carbonaceous vapor 10 (as the glycerol vapor) sent in the plasma zone. The vapor reacts with discharge and oxidizer to produce a partly reformed gas. The plasma head can also include an exit 23. The partly reformed gas can take out the plasma head by the exit 23 and can then react with the catalyst 17 put in the post plasma zone 14b to form the synthesis gas. The temperature at the exit 23 and so in the entrance of the zone 14b can be included between around 900 and 1250° C.

The present invention concerns therefore first, a method to convert carbonaceous matter into the synthesis gas. This method includes the preheating of an oxidizer into a predetermined temperature, an excitation of this oxidizer preheated in a zone of plasma vortex, and an exposure of carbonaceous matter to the preheated and excited oxidizer. The method also includes the reaction of the carbonaceous matter contacted with the oxidizer preheated and excited in the high voltage discharge to produce a partly reformed gas in the plasma zone and fact to react the partly reformed product with a catalyst to form the synthesis gas in a post plasma zone.

The total reformation process of carbonaceous material of the general formula CₓH_{y}O_{z} may be described schematically by two chemical reactions:

CₓH_{y}O_{z}+(x−z)/2O₂=x CO+y/2H₂ (5)

CₓH_{y}O_{z}+(x+y/4−z/2)O₂=x CO₂+y/2H₂O. (6)

The principal reaction (5) of partial oxidation is weakly exothermic and cannot, by itself, form enough heat necessary for the conversion. In other words, the principal reaction cannot heat the reactor and its contents to a sufficient temperature, that is to say at least 900° C. (but not higher than 1250° C.). These temperatures are necessary for sufficiently rapid kinetics in the presence of a mildly active catalyst 17. The complete combustion reaction (6) does not give any desirable products, but is highly exothermic. In combining the two reactions in a good proportion of “p” parts for reaction (6) and (1−p) parts for reaction (5), one can find a compromise of sufficiently high selectivity of production of syngas at sufficiently high temperatures that promote the kinetics of reaction (5) in the presence of a mildly active (and therefore simple and resistant to deactivation) catalyst. The proportion p is kept in the range of 0.1 to 0.3 (or in other words 10 to 30%) that defines the ratio of the oxygen to the organic molecule to convert. For example, for glycerol the principal reforming reaction is:

C₃H₈O₃=3CO+4H₂. (5a)

The total combustion is:

C₃H₈O₃+3.5O₂=3CO₂+4H₂O. (6a)

For the proportion p=0.2 (20%) the combined process is therefore:

C₃H₈O₃+0.7O₂=2.4CO+3.2H₂+0.6CO₂+0.8H₂O. (7)

C₃H₈O₃+3.3 air=2.4CO+3.2H₂+0.6CO₂+0.8H₂O+2.6N₂ (7a)

It is evident that (1−p)*100 represents the selectivity (in this case 80%) of the conversion of glycerol to syngas, realized in mixing 0.7 moles of oxygen and 1 mole of glycerol. If air (containing 21 vol. % O₂) is used as the source of oxygen, the last reaction may be rewritten as:

The zone 14b is thermally insulated from the exterior by the jacket 16 to limit the loss of heat. The jacket acts at the same time as a pre-heater for the oxidant 15a. Consequently, the factor p is reduced and the quantity of syngas produced is therefore raised. It is preferable not to cool any part of the reactor 14a and 14b.

As shown in FIG. 2, the two compartments 14a and 14b are placed in series with respect to the flux of reactants and products. The first phase of the conversion, partial oxidation of the carbonaceous matter, is already started in the plasma zone. In this zone, an oxidation process (6a) is observed for the matter in its vapor phase.

We have observed that if the matter is in its liquid phase, even dispersed, the majority of the droplets cross the zone 14a without being oxidized. Such a very hot and heterogeneous mix is projected on the first catalyst 17 layer where the droplets can partially pyrolyze, giving some soot or coke that blocks the contents of the zone 14b. That is why it is imperative that the carbonaceous matter to convert be almost entirely in the vapor phase.

Practical realization of process like (7) or (7a) is executed in the device schematically illustrated in FIGS. 2 and 3. It is well known that the glycerol (as well as certain other organic liquids) is difficult to ignite in the ordinary conditions (air as the oxidant, atmospheric pressure, and ambient temperature). These can only be ignited and converted to syngas at an elevated temperature (above about 600° C.). The first thing to do is therefore preheat the reformer. This is done by using an easily flammable gas (for example, natural gas, methane, propane) sent into the head 14a while an oxidant 15b, such as air for example, enters in the same head by a separate entrance. Evidently this mix has to be ignited. It is preferable that a vortex and gliding electric discharge is used, this as described above.

When the sensor S that is the closest to the intersection between 14a and 14b indicates a sufficiently high temperature (we want it between 900 and 1250° C. approximately), the igniting fuel flow rate is progressively reduced and at the same time, the flow rate of carbonaceous material to convert is considerably increased. During this operation, the temperature is preferably kept in the aforementioned range. It is preferable to keep these temperatures in the range by controlling the ratio R of oxygen to igniting fuel plus the charge to convert. The ratio R is raised when the temperature is decreased, and vice versa. At the end of the reformer start-up, the preheating fuel is stopped. The oxidant flow rate can then be progressively increased as well as the flow rate of the matter to convert, this is to keep the reformer operating temperature in the range previously stated: between about 900 and 1250° C. In increasing the oxidant and feed flow rates (then by charging the reformer more and more), a given temperature (in the stated range) may be obtained for a smaller ratio R, and therefore a smaller p, this is for a better selectivity of syngas generation. The composition of syngas (and therefore its quality and applicability for a given use) for a given reformer volume, depends on several variables. These variables can, for example, include:
f) The composition of the feed,
g) The composition of the oxidant,
h) The ratio R, or the parameter,
i) The reformer charge (the ratio of feed to the volume of the zone 14b), and
j) The properties of catalyst 17 present in the post-plasma zone 14b.

It is therefore essential to analyze online the syngas composition as a function primarily of the variables c) and d) for the given constants a), b) and e). When the composition of syngas is acceptable, the reforming parameters can be established and then used as a scenario for a simple and efficient control process for the wide range of desirable feedstocks (and therefore, the quantities and properties of syngas).

The principal processes (7) or (7a) happen in the zone 14b filled with a catalyst. The metallic nickel and its oxides are present at the surface and in the interior of a porous material. As described before, the flux of partially reacted gas still contains certain non-reacted molecules from the initial carbonaceous material, as well as an excess of CO₂ and water vapor at a temperature between about 900 and 1250° C. In the presence of a catalyst, two endothermic processes of dry reforming and steam reforming happen as follows:

CₓH_{y}O_{z}+CO₂→CO+H₂ (8)

CₓH_{y}O_{z}+H₂O→CO+H₂ (9)

Progressively, the non-reacted matter disappears by pumping the heat from its hot environment. As a result, the temperatures decrease and the completely reacted gas 12a leaves the last catalyst layer of zone 14b and then an exit tube at moderate temperatures varying between about 300 and 500° C. The total process can therefore be described by reactions (7) or (7a) without the initial over-oxidation of a part of the matter followed by the dry- and/or steam-reforming (8) and (9), respectively. Still another process happens in zone 14b—“water-gas shift”:

CO+H₂O═CO₂+H₂ (10)

This reaction forms an excess of hydrogen and a deficit of carbon monoxide with respect to the ratio of concentrations calculated based on reactions (7), (7a) and the parameter p. For certain specific applications a supplemental quantity of H₂ may be of interest. For example, the molar ratio of H₂/CO based on reaction (7) should be equal to about 1.3, which is insufficient to lead the Fischer-Tropsch process according to the paths (1) or (2), but the process (10) can, at least partially, remedy this.

The reactions (8), (9) and (10) are accelerated by the same catalyst 17. A catalyst precursor filling zone 14b becomes active with its first use. During the process start-up, the entire reformer is heated in these oxidizing conditions (for example, in an excess of air). In these conditions, all the nickel and its compounds (regardless of their initial chemical formula) become oxidized. The nickel oxides, such as, for example, NiO, Ni₃O₄, and Ni₂O₃, change from one to the other as a function of the temperature and the availability of oxygen. This property is used for our catalytic process of partial oxidation of the carbonaceous matter. The total process conversion (100%) of this matter (not a trace of it found in the syngas) is explained by the following reasons:

The metallic nickel and its oxides NiOₓ (general formula) are initially on the surface of (and inside) the porous grains in zone 14b, reacting with certain molecules present in the zone. Two cases can be distinguished as follows: The oxides and/or metallic nickel increase their oxidation state, for example:

Ni+H₂O═NiO+H₂ (11a)

Ni+CO₂═NiO+CO (11b)

2NiO+H₂O═Ni₂O₃+H₂ (11c)

2NiO+CO₂═Ni₂O₃+CO (11d)

or they reduce their oxidation state when in contact with the initial organic vapors and/or certain molecules issued from pyrolysis, for example:

2Ni₂O₃+C₂H₄=4NiO+2CO+2H₂ (12a)

2NiO+C₂H₂=2Ni+2CO+H₂ (12b)

NiO+C═Ni+CO (12c)

NiO+tar→Ni+CO+H₂ (12d)

The nickel and its oxides therefore become the very active donors and receptors of oxygen. These cyclic oxidation/reduction processes drive the conversion of the entire quantity of carbonaceous material to the desired syngas products.

It could happen, in the case of start-up errors, that a massive, undesirable pyrolysis of the matter happens in zone 14b. In this case the soot and/or coke and/or tar form. These products can pollute the syngas. Furthermore, the soot and coke, according to our study, catalyze the pyrolysis itself, which provokes an increase of the pressure drop on the reformer. Finally, after a certain time, the entire zone 14b becomes blocked. When such an undesirable process begins to happen and, in the same time, a sensor S placed as close as possible to the intersection between 14a et 14b still indicates a temperature of at least 700° C., it is possible to regenerate/clean the catalyst 17 by stopping the feeding of the carbonaceous matter to be converted and by reducing the oxidant input. In such conditions, an intensive oxidation of metallic nickel and its lower oxides and all other products can be observed. One can allow the temperature of the bed to reach 1250° C., by controlling the amount of oxidant. When the first catalyst layer is cleaned, the process progresses rapidly, because of the progressive heat transfer. The temperatures become high enough for progressive cleaning of deeper layers of polluted catalyst. For such a complete cleaning operation, “layer by layer” of the entire zone 14b, one can reactivate the catalyst and start to reform again the feed. It is preferable to keep the temperature of the oxidation of mobile layers only up to 1250° C. to avoid destruction of the catalyst.

When the sensor S indicates a temperature below 700° C., the mobile cleaning process cannot start, but may still be done by proceeding to a similar operation at cold start-up. One sends a little bit of start-up fire gas and the oxidant in the presence of the electric discharge to heat the first layer of zone 14b. When the sensor S indicates at least 700° C., one can stop the gas and proceed to the cleaning described in the preceding paragraph. The reforming process may be active for several days. If one observes a progressive deactivation of the catalyst (due to soot/coke deposits)—one can proceed to the reactivation of the catalyst as described above.

Here are some results that we obtained during our trials in a laboratory scale:

### Trials 1

Reformer having a zone 14b volume of 0.57 L. Electrical power of discharge: 50 W.
Carbonaceous entry matter: 91% liquid glycerol, 9% water; Lower Heating Value (LHV) of 4.1 kWh/kg. Dosing by a pump in the range of 1-16 g/min.
Oxidizer: a mixture of air/O₂ containing 21-61 vol. % O₂; air flow rate: 4.6-14 L(n)/min; O₂ flow rate: 0-4.8 L(n)/min.
Auxiliary fuel: natural gas; flow rate: 0.5 L(n)/min corresponding to LHV of 0.3 kW.
As results of conversion, we get:
Syngas output flow rate (dry): 17-30 L(n)/min; its LHV: 1.0-2.0 kWh/m³(n).
Some details of the Test 8:
   Entry: 14.9 g/min glycerol+0.8 L(n)/min natural gas 4.6 L(n)/min air+1.7 L(n)/min O₂
   Exit: 28.5 L(n)/min syngas containing 16.3% CO₂, 0.3% C₂H₄, 28.5% H₂, 2.3% CH₄, 14.2% CO, 38.3% N₂
      LHV of the syngas: 1.6 kWh/m³(n)
      Chemical (LHV) power of the syngas: 2.8 kW.
         This syngas contains 43 vol. % of H₂+CO in a molar ratio H₂/CO=2. Such syngas can therefore be considered also as reactant for the FT synthesis of clean liquid fuels (especially the Diesel fuel).

### Trials 2

Reformer having a 14b zone volume of 1.4 L. Electrical power of discharge: 50 W.
Carbonaceous matter entry: 97% glycerol; its LHV of 4.3 kWh/kg; dosing the liquid by a pump in the range of 13-52 g/min; the liquid is vaporized in an electrical oven before being injected into the plasma head 14a.
Auxiliary fuel: natural gas; flow rate: 0.5 L(n)/min corresponding to LHV of 0.3 kW.
Oxidizer: air; flow rate: 25-54 L(n)/min
Results of conversion: Flow rate of syngas (dry): 26-127 L(n)/min; LHV: 1.1-2.0 kWh/m³(n). Chemical (LHV) power of the syngas: 1.9-14 kW.

Now let us take into account a balance of matter and energy for a process where 1.00 kg of pure liquid glycerol is heated from a temperature of 25° C. up to its boiling point of 290° C. under the pressure of 1.013 bar, and then it is entirely evaporated in the same temperature and pressure. The spent enthalpy will be 1.29 MJ. Let us imagine that we succeed in burning 1.00 kg of pure liquid glycerol and, that all its LHV of 1.48 MJ/mol is recovered, which is equivalent to 16.1 MJ. We note that the ideal combustion of 1.00 kg of the glycerol can give the enthalpy for evaporation of 12.5 kg of the glycerol taken initially at 25° C. under atmospheric pressure. In other words, to generate 1.00 kg of the glycerol vapor on these conditions, it is necessary to dispose of 1.08 kg of the liquid glycerol from which of only 0.08 kg are taken to burn it.

In practice, the energy efficiency of the conversion of the glycerol vapor into synthesis gas before described, is (or exceeds) 80%. It means that for 1 kg of entering glycerol vapor, 20% remaining (or less) LHV is converted to heat, equal (or exceeding) to 3.2 MJ and contained in the syngas 12a going out of the zone 14b at a temperature of at least of 350° C. This quantity of heat is enough for bringing to the vapor state at least 2.5 kg of the liquid glycerol. Operation before recalled as Step C can be judiciously performed in devices 9 and/or 13. Of this 2.5 kg of vapor 1.0 kg is then taken for the conversion into syngas. Remaining 1.5 kg of glycerol vapor can be condensed selectively or in a “batch” way into liquid glycerol 7 for a valorization on a distant industrial site.

Let us add that at least 80% of the initial LHV entering glycerol is in the form of the chemical enthalpy of the exiting syngas. This syngas can easily feed a turbine, an internal or external combustion engine, a fuel cell, etc.—this to generate the electrical or mechanical power. This syngas can also be considered to be a reactant for numerous chemical syntheses. But the most attractive use of such syngas is, to our opinion, in the FT synthesis of renewable and clean liquid fuels capable to replace aforementioned fossil fuels.

Let us therefore return, once again, to the FT technique. Heat produced by a classical FT unit serves for generating the water steam at a medium pressure, which can drive a turbine producing electricity, which would participate in the electrical supply of the site of conversion of the natural gas, of the biogas or of other gases coming from a waste treatment of biomass—into clean fuels. And why not to generate the glycerol vapor?

Reaction schemes (1-3) point out that their balances are moved to the right by increasing pressure. For example, if reaction (1) is complete, the shrinkage of the system is defined by the ratio of the total number of moles of gaseous products (n+1) to the total number of moles of gaseous reactants (3n+1). This report is therefore equal to 0.32 for the examined molecule C₁₀. It points out that a better conversion rate of the syngas into synthetic fuel (Syncrude) can be obtained under more and more high pressure in a reactor of constant size. By increasing pressure in a FT catalytic bed and, by augmenting the activity of the catalyst, it would be possible to reduce the size of the FT reactor for the same aimed productiveness if only one succeeded well in a better heat removal when the FT synthesis takes place inside the tubes. Let us not forget that, warmed up by FT synthesis, a granule of FT catalyst which is in the axis of the tube and which is distant from the wall of the tube cooled down outside by the water and/or the steam, classical solution, must export its portion of heat on a path often larger than 1 cm through other granules. The classical catalysts, on mineral support in the order of 5 mm, are compatible with the conception of the available industrially reactors. The disadvantage of the classical catalysts is related to their relatively low thermal conductivity. This disadvantage can contribute to a local overheating and, therefore to the starting of the parasitic reaction (4) which, also, is favored by the increase of pressure, because the previously defined rate of its shrinkage is equal to 0.5. Is it then possible to fix and to support constant the optimum temperature of such “central” grain as well as the “peripheral” grains close to the wall of the tube? Is it possible to avoid a catastrophic deposit of coke?

A solution was already offered in our French patent No 2824755 (A. Czernichowski and M. Czernichowski, Réacteur à plaques et son fonctionnement dans un procédé catalytique exothermique). The invention describes a compact reactor with numerous strongly gripped and piled plates. It also specifies the way to accomplish, with this reactor, strong exothermic reactions, such as FT synthesis in the presence of grains of a fine catalyst filling the channels with said reactive plates R. Other metallic neighboring plates, say heat transporting plates C, of a size and similar form, are cooled by a heat medium. Plates C are strongly gripped on both sides of every plate R to assure a very good thermal contact between them. All stacking up of plates R and C is easily knockdown to be able to easily perform a replacement by a part of reconditioned plates R. Unfortunately, this solution seems too much ahead of time for rather conservative industrial world. At present almost all FT reactors with fixed bed are still constructed as they were more than 70 years ago! Even any new plans still envisage these same reactors.

Productiveness (defined, for example, as a ratio of the quantity per hour of the produced syncrude to the volume of the reactor), capital and operation costs, energy output, weight, and security of a reactor FT are a complex function dependent on several parameters. The most important are:
Activity of the catalyst put in tubes: for a given catalyst, it augments exponentially with the temperature (up to some limit of given catalyst and desired FT products). Some very interesting catalysts from the point of view of their selectivity, longevity, poisoning resistance, etc. become active only in relatively high temperatures, inaccessible when the water steam is used as the heat medium (coolant).
The maximum temperature (and very often optimum at the same time) of the catalyst is directly linked to the maximum temperature of the fluid cooling the tubes of the reactor (the water steam in the majority of cases).
Carnot's energy efficiency augments with the temperature of the hot source (cooling fluid) in comparison with the cold source (for example, the external environment of the reactor): this point concerns a valorization of the heat produced by FT technique.
The saturating vapor pressure of the reactor cooling fluid is almost exponential function of its temperature.
Cost, security, weight, and size of the reactor are directly linked to the thickness of the external surrounding wall of the vessel sheltering the tubes, this thickness being a very strong function of pressure inside the vessel.

FIG. 4 introduces pressure (in absolute bar) of saturating water steam and of the glycerol according to “Handbook of Chemistry and Physics, 81st and 44th Edition”. One sees there, that to bring the temperature of the surrounding wall of tubes to 290° C., it would be necessary to pressurize the vessel up to rather excessive 74 bar of the water steam while, at this boiling point of pure glycerol, the pressure is only 1 bar. If tubes were cooled using pure glycerol, one would have an access to temperatures until 400° C., for which pressure in the vessel would be only of 13.6 bar, while, for the water at 370° C., the pressure exceeds already 220 bar! Although the water is known for its exceptional high specific heat of 4.18 J/(g·K), the glycerol presents 57% of this value, what would require augmenting the debit of circulatory heat fluid between the vessel and an external heat exchanger (cold source) by a factor of only 1.7 in comparison with the water. But is it imperatively necessary to circulate all glycerol between the vessel and the exchanger?

On the FIG. 1, we add therefore this integrated solution of circulation of matter and of energy, described above.

We prefer sending a part of glycerol vapors 10 going out of the vessel E directly, as matter to be converted into syngas, to the plasma zone 14a of our reformer. So we accomplish a flexibility of feeding the reformer in vapor 10 of carbonaceous matter: either from batch distillation 9 of the raw glycerine 5 or from FT unit. Naturally, for a reason of suppleness and of management of fluids, a part of vapors 10 going out of the FT reactor is condensed in a heat exchanger 24 being in contact with a classical heat medium 25. The glycerol condensed in the exchanger serves once again as heat medium 7 for the FT reactor. To this fluid, it is naturally necessary to add the pure glycerol 7 coming, for example, of the operation 9 of the batch distillation of the waste glycerine. Such glycerol can contain some water or other liquids making its boiling point inferior to that of pure glycerol—what can contribute to an increase of pressure within the FT vessel. That is why we give a preference to the pure glycerol coming from a vacuum distillation (or a fractionated distillation) operated in 6.

The circulation of heating media 4, 7, 11, and 25 is not a part of this description. It is under the control of pumps, pressure gauges, valves of regulation, gates of insulation, flow controllers, and of quite other equipment allowing managing these fluids. Heats coming from reactions (1-3) and (5-12d) here described are sent towards fluids circulating according to the FIG. 1. The optimized management, these circuits are assured via a control of temperatures, with a precision of at least ±5° C. and preferably ±1° C.

The heat medium 25 can therefore have a higher temperature in comparison with a classical water steam FT reactor. This heat medium can be used in a thermodynamic machine to produce a noble energy (mechanic and/or electric) required to feed pumps, compressors, presses of oil, etc. The Carnot's energy efficiency of such operation will therefore be higher because the temperature of heat medium can be raised (for example 330° C. instead of 230° C. for a classical FT reactor). A residual heat of Carnot's operation can finally find a usage included on the industrial site (for example, as heat 4 to recover the methanol in the primary raw glycerine 2 going out of operation 1). The heat produced by a FT unit cooled by glycerol would serve, for example, for generating much higher-pressure (HP) water steam than the steam produced in a classical FT reactor. Such HP steam drawing a turbine would give more electricity supplying here proposed “Integrated Plant of Biodiesel and Syncrude”.

1. A technique for cooling of a reactor with a fixed catalytic bed for exothermic catalytic syntheses, wherein the exothermic catalytic syntheses is a Fischer-Tropsch (FT) synthesis of liquid fuels by selective hydrogenation of monoxide of carbon (CO) by hydrogen (H₂), the mixture of these two gases being defined as “synthesis gas” or “syngas”, the technique being characterized by:
heat of the synthesis being taken away by a heat medium of non-fossil origin and having a boiling point higher than the boiling point of water; and
at least a part of vapors that result from the boiling heat medium being extracted from the reactor to feed, as a carbonaceous matter, another reactor being defined as a “reformer”, in which the vapor is converted into syngas to feed the aforesaid reactor of exothermic synthesis.

2. A technique according to the claim 1 wherein the heat medium is glycerol being a co-product of at least one of biodiesel and soap production.

3. A technique according to the claim 1 wherein the vapor coming from the heat medium that cools the catalytic exothermic reactor is converted into synthesis gas through a partial oxidation reaction and wherein another gaseous reactant of oxidation, defined as an oxidizer, contains elementary oxygen O₂.

4. A technique according to the claim 3 wherein the oxidizer contains between 21 and 100 percent by volume of oxygen O₂ and wherein the vapor of carbonaceous matter comes from the glycerol.

5. A technique according to the claims 1 or 3 wherein the quantity of available elementary oxygen in a flux of the oxidizer does not exceed 30% of a quantity necessary for a complete combustion of the matter.

6. A technique according to the claims 1 or 3 wherein the heat medium vapor and the oxidizer are introduced separately into a plasma device of a reformer and wherein the oxidizer is preheated by a residual heat produced during partial oxidation, and wherein the reaction of oxidation is accomplished in an adjacent part of the reformer filled with a catalyst that is connected in series to the plasma device.

7. (canceled)
